(19) United States
(12) Patent Application Publication
(10) Pub. No.: US 2024/0366489 A1
(43) Pub. Date: Nov. 7, 2024

(54) NAIL POLISH REMOVER COMPRISING GLYCOLIPIDS

(71) Applicant: LOCUS SOLUTIONS IPCO, LLC, SOLON, OH (US)

(72) Inventors: LEE SPEIGHT, SOLON, OH (US); AYENDA ZEMAITIS, MIDLOTHIAN, VA (US); DANIEL HAGAMAN, MECHANICSVILLE, VA (US); SEAN FARMER, FT. LAUDERDALE, FL (US)

(21) Appl. No.: 18/248,380

(22) PCT Filed: Jan. 28, 2023
(86) PCT No.: PCT/US2023/061519

§ 371 Date: Apr. 8, 2023

Related U.S. Application Data
(60) Provisional application No. 63/304,104, filed on Jan. 28, 2022.

(51) Int. Cl. A61K 8/60; A61K 8/34; A61K 8/35; A61K 8/37; A61K 36/06; A61Q 3/04

(52) U.S. Cl. CPC A61K 8/60; A61K 8/345; A61K 8/35; A61K 8/37; A61K 36/06; A61Q 3/04

(57) ABSTRACT

In one aspect, the invention pertains to a nail polish remover composition comprising a glycolipid to reduce the occurrence of dry and/or irritated skin as well as dry nails adjacent to the nails, and/or hydrate the skin and nails. The presence of glycolipids in the composition further facilitates wetting of the nail care product and removal of the product from the nail. In another aspect, the present invention pertains to a treatment composition and method of using such composition to hydrate nails and surrounding skin and/or treat a fungal infection of hands and feet.

### CROSS-REFERENCE TO A RELATED APPLICATION

This application claims priority to U.S. provisional patent application No. 63/304,104 filed Jan. 28, 2022, the entire contents of which are incorporated herein by reference.

### BACKGROUND OF THE INVENTION

Nail care is one of the most important aspects of personal grooming, and its market has been steadily growing. Among popular nail care products, nail polish (also called nail varnish or nail enamel) is a lacquer that is applied to fingernails or toenails to temporarily decorate, color, coat, and/or protect the nails. The practice of lacquering nails with nail polish has been around for centuries. Additionally, artificial nails have become prevalent in the recent years. Artificial nails come in variety of forms including acrylic nails, gel nails, nail wraps (e.g., silk, linen, or fiberglass wraps), nail tips, and dip powder. They are generally applied or bonded to natural nails. Typically, application of artificial nails is followed by application of nail polish.

As part of regular maintenance, these nail care products are frequently removed, commonly with commercially available solvents in the form of nail polish removers. For example, artificial nails are often removed by placing a piece of cotton saturated with nail polish remover on each of the artificial nails, then wrapping a tin or aluminum foil over the nails. This setup is kept in place until the polish remover thoroughly wets and causes the artificial nails to soften or separate from the natural nails. In some instances, the fingertips are dipped directly in a bowl filled with nail polish remover or in a remover-soaked sponge. A typical process of removing lacquers of nail polish from natural or artificial nails involves repeated rubbing of the nails with an absorbent material (e.g., a cotton ball) saturated with nail polish remover until the lacquers dissolve and are removed from the nails. Other methods of nail polish removal include soaking the fingertips in a bowl filled with remover in the manner similar to the removal process for artificial nails before the nail polish can be wiped off the nails.

A typical nail polish remover composition is a liquid that comprises an organic solvent as the active ingredient and may also include oils, scents, and colorings. Common active ingredients include acetone, acetates, and/or alcohols. Unfortunately, these active ingredients can be damaging to the skin, leaving the skin appear red, dry, and/or cracked, diminishing the aesthetic effects of nail care products. The problem is exacerbated when the remover must come in contact with the skin repeatedly or for a prolonged time, for example, during the various removal processes described above.

Numerous attempts have been made to develop a nail polish remover formula that reduces skin drying and irritation, typically by using milder active ingredients and/or moisturizers. However, many such formulas have undesirable side effects including less efficient removal power and greasy residues left on the skin after use. Such problems necessitate added steps in the removal process, often involving extra tugging or rubbing of the nails (and consequently the surrounding skin) to completely wipe off the grease or nail lacquer. This can inadvertently damage the nails and the surrounding skin. If a remover formula is strong enough to remove nail polish lacquers but not artificial nails, a different and harsher formula is still needed, causing the affected skin and/or nails to be dry and/or irritated.

Accordingly, there is a need for a nail polish remover composition that reduces the occurrence of skin and nail drying and/or irritation while facilitating efficient removal of nail care products.

Moreover, in order to keep the nails and the surrounding skin looking their best, maintenance of their health is essential. Keeping the skin and nails moisturized not only provides aesthetic benefits, but also renders those areas less susceptible to skin infections, as they become more pliable and less at risk for cracking, tearing and hangnails. Thus, it would be advantageous to provide a composition that can hydrate dry skin and nails such that application of such composition increases their water content compared to before the application.

Fungal infections, or mycosis, are most common on the skin or nails. Onychomycosis is a fungal infection of the fingernails or toenails that causes discoloration, thickening, onycholysis (separation from the nail bed), and nail plate dystrophy. It is mainly caused by yeast, dermatophytes, or other nondermatophyte molds, with dermatophytes being the most common cause. Onychomycosis affects an estimated 10% of the general population, although it is more common in older adults. Up to 35 million Americans are estimated to have onychomycosis, and onychomycosis represents approximately 50% of all nail disorders. Toenail infection accounts for approximately 80% of onychomycosis incidence.

Another type of mycosis is a fungal infection of hands and feet, termed tinea manuum and tinea pedis, respectively. These diseases, often referred to as ringworm, affect the outer layer of the skin, and are characterized by itchy, round patches that may develop into a series of rings with central clearings. The affected skin may thicken. become intensely dry, and/or develop deep cracks, white scaling, or pain and swelling. Frequently the infection spreads to the fingernails close to the affected skin. Tinea pedis is also called athlete's foot, which affects soles, interdigital clefts of toes, and nails with a dermatophyte fungus, which usually begins between the toes. Tinea manuum and tinea pedis are the most frequently diagnosed fungal infections, with tinea pedis occurring in up to 70% of adults, while tinea manuum is the diagnosis in up to 13% of fungal infections.

These diseases adversely affect the quality of life of their victims, with complaints ranging from unsightly hand and nails as well as discomfort with footwear, to more serious complications including secondary bacterial infections. A composition that can conveniently treat such conditions is highly desirable.

### BRIEF SUMMARY OF THE INVENTION

In one aspect, the present invention provides nail polish remover compositions comprising a removal solvent and a glycolipid. The composition may further comprise a glycol co-solvent, water, and/or suitable additional components that do not adversely interfere with the functions of the removal solvent and the glycolipid.

Another aspect of the present invention provides methods of removing a nail care product from a surface comprising applying a composition comprising a removal solvent and a glycolipid, and removing the nail care product from the surface. The surface may be a bodily surface or a non-bodily surface. The composition may further comprise a glycol co-solvent, water, and/or suitable additional components that do not adversely interfere with the functions of the removal solvent and the glycolipid.

In some embodiments, the glycolipid in the nail polish remover composition is a rhamnolipid, a sophorolipid, a mannosylerythritol lipid, and/or a trehalose lipid. More preferably, the glycolipid is a sophorolipid and/or a mannosylerythritol lipid.

According to the present invention, the nail polish remover composition of the present invention protects nails and/or skin surrounding the nails from damaging effects of a removal solvent. It helps to reduce the occurrence of dry and/or irritated skin after the composition has been applied to the skin. In some embodiments, the composition similarly helps to reduce the occurrence of dry nails. Furthermore, the glycolipids facilitate penetration or “wetting” of the nail care products to be removed by the nail polish remover composition, thereby enabling more efficient removal of the nail care products.

In other aspects, the present invention provides a treatment composition for skin and nails, and a method of moisturizing the skin and nails using same.

In yet other aspects, the present invention provides a treatment composition for treating a fungal infection of hands and feet, and a method of treating a fungal infection of hands and feet using same. In specific embodiments, the fungal infection of hands and feet includes onychomycosis and tinea pedis.

### DETAILED DESCRIPTION OF THE INVENTION

The present invention provides nail polish remover compositions and methods for removing nail care products from a bodily or non-bodily surface. The present invention further provides treatment compositions for hydrating the skin and nails, and/or treating a fungal infection of hands and feet. The nail polish remover compositions and treatment compositions may be used by itself, concomitantly, or sequentially, depending on the need of a subject.

### Definitions

The term “nail polish remover” as used herein generally refers to a composition capable of interacting with a component of a nail care product disposed on or otherwise attached to a bodily or non-bodily surface in such a way that the composition solubilizes, dissolves, weakens, loosens, or otherwise disrupts the component thereby permitting removal of the nail care product from such surface. In one aspect, the bodily surface may be a fingernail, a toenail, and/or skin surrounding the nail. The non-bodily surface may be, for example, an artificial nail, a countertop, or a floor. Thus, purposes of using the nail polish remover include, but are not limited to, removal of a nail polish lacquer from natural or artificial nails. Other purposes may be to remove artificial nails from natural nails. Yet other purposes may be to clean countertops or floors, for example, a spill of a nail care product on such surfaces.

It will also be understood that, while the nail polish remover composition according to the present invention is capable of removing nail polish from a surface, its use is not necessarily limited to removal of nail polish or other nail care products. For example, in accordance with certain embodiments, the nail polish remover composition is used to simply clean the nails and/or other parts of the body to prepare such surfaces for new application of a nail care product or other cosmetics. Other non-limiting, exemplary uses of the nail polish remover composition include removal of transfer-resistant cosmetics (e.g., smudge-proof lip stains), superglue, and marker or paint stains from bodily or non-bodily surfaces.

The term “nail care product” encompasses products that are designed to be applied to or adjacent to nails and temporarily remain there, and primarily for cosmetic purposes. Non-limiting examples of the nail care product include nail polish (including nail strengthening polish, base coat, top coat) and artificial nails. Other non-limiting examples include cuticle oil, hand cream, and anti-chew treatment.

The term “bodily” concerns the body of an organism, including mammals such as primates. Mammalian species that can benefit from the disclosed compositions and methods include, but are not limited to, apes, chimpanzees, orangutans, humans, and monkeys; domesticated animals such as dogs, cats; live stocks such as horses, cattle, pigs, sheep, goats, and chickens; and other animals such as mice, rats, guinea pigs, and hamsters. Preferably, the bodily surfaces to which the nail polish remover composition of the invention is applied are human bodily surfaces, such as fingernails, toenails, and/or the surrounding skin.

The term “subject” as used herein, describes an organism, including mammals such as primates. Mammalian species that can benefit from the disclosed methods of treatment include, but are not limited to, apes, chimpanzees, orangutans, humans, and monkeys; domesticated animals such as dogs, cats; live stocks such as horses, cattle, pigs, sheep, goats, and chickens; and other animals such as mice, rats, guinea pigs, and hamsters. In some embodiments, the subject is a mammal. In further embodiments, the subject is a human.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention, e.g., the ability to improve the bioavailability of a substance. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example, within 2 standard deviations of the mean. As further examples, “about” can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Nail Polish Remover Composition

### Glycolipids

In one aspect, the present invention relates to a nail polish remover composition comprising a glycolipid. In certain embodiments, the amount of a glycolipid in the nail polish remover composition according to the present invention is at least about 1% by weight, preferably between about 1% by weight and about 5% by weight of the total nail polish remover composition.

Glycolipids are lipids that are covalently attached to a carbohydrate via a glycosidic bond. Glycolipids as used herein are naturally derived compounds of a microbial origin as metabolic by-products that exhibit surfactant properties (emulsification capabilities and a reduction in surface/interfacial tension). They are commonly classified based on their biochemical nature and/or by the species of microbe that produces them. Glycolipids are safe, biodegradable, and can be produced with ease at low cost using selected organisms in or on renewable substrates. Glycolipids according to the present invention can increase moisture retention, lubrication, or smoothness of skin, and/or minimize the occurrence of dryness, irritation, and/or wrinkles. Similarly, the glycolipids according to the present invention can increase moisture retention and smoothness of nails.

Glycolipids are types of biosurfactants that are amphiphiles and consist of two parts: a polar (hydrophilic) moiety and non-polar (hydrophobic) group. Due to their amphiphilic structure, biosurfactants increase the surface area of hydrophobic water-insoluble substances, increase the water bioavailability of such substances, and change the properties of bacterial cell surfaces. Furthermore, biosurfactants accumulate at interfaces, and reduce the surface and interfacial tension between the molecules of liquids, solids, and gases, thus leading to the formation of aggregated micellular structures in solution.

Most biosurfactant-producing organisms produce biosurfactants such as glycolipids in response to the presence of a hydrocarbon source (e.g., oils, sugar, glycerol, etc.) in the growing media. Other media components such as concentration of iron can also affect biosurfactant production significantly. Microbial biosurfactants are produced by a variety of microorganisms, such as, for example, Pseudomonas spp. (P. aeruginosa, P. putida, P. florescens, P. fragi, P. syringae); Flavobacterium spp.; Bacillus spp. (B. subtilis, B. pumillus, B. licheniformis, B. amyloliquefaciens, B. cereus); Wickerhamomyces spp. (e.g, W. anomalus), Candida spp. (e.g., C. albicans, C. rugosa, C. tropicalis, C. lipolytica, C. torulopsis); Rhodococcus spp.; Arthrobacter spp.; Campylobacter spp.; Cornybacterium spp.; Pichia spp. (e.g., P. anomala, P. guilliermondii, P. occidentalis); Starmerella spp. (e.g., S. bombicola); and so on.

The biosurfactants including glycolipids may be obtained by fermentation processes known in the art, e.g., solid-state fermentation, submerged fermentation, or modifications, hybrids, and/or combinations thereof. The production of biosurfactants with the use of renewable substrates and different microbial species, as well as the variation in culture parameters (incubation time, stirring speed, ph of the medium and added nutrients), allow for the acquisition of compounds with distinct structural and physical properties. This makes it possible to produce a wide variety of compounds that can elicit different physical, chemical, biochemical, and biophysical properties.

The microorganisms of interest can be cultivated at small or large scale on site and utilized, even being still-mixed with their media. Similarly, the microbial metabolites (e.g., glycolipids) can also be produced at large quantities at the site of need. The growth vessel used for growing biosurfactant-producing organisms can be any fermenter or cultivation reactor for industrial use. In one embodiment, the vessel may have functional controls/sensors or may be connected to functional controls/sensors to measure important factors in the cultivation process, such as pH, oxygen, pressure, temperature, agitator shaft power, humidity, viscosity, and/or microbial density and/or metabolite concentration.

In one embodiment, a single type of microorganism is grown in a reactor system. In alternative embodiments, multiple microorganisms, which can be grown together without deleterious effects on growth or the resulting product, can be grown in a single reactor system. There may be, for example, 2 to 3 or more different microorganisms grown in a single reactor at the same time. In some embodiments, more than one microorganism grows symbiotically in the reactor.

The cultivation can be supplemented with a nitrogen source. The nitrogen source can be, for example, potassium nitrate, ammonium nitrate, ammonium sulfate, ammonium phosphate, ammonia, urea, and/or ammonium chloride. These nitrogen sources may be used independently or in a combination of two or more.

The growing culture in the cultivation can also be oxygenated. One embodiment utilizes slow motion of air to remove low-oxygen containing air and introduce oxygenated air. In the case of submerged fermentation, the oxygenated air may be ambient air supplemented daily through mechanisms including impellers for mechanical agitation of the liquid, and air spargers for supplying bubbles of gas to the liquid for dissolution of oxygen into the liquid.

The cultivation may be supplemented with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, isopropyl, propanol, butanol, pentanol, hexanol, isobutanol, and/or glycerol; fats and oils such as soybean oil, rice bran oil, canola oil, olive oil, corn oil, sesame oil, and/or linseed oil; etc. These carbon sources may be used independently or in a combination of two or more.

In one embodiment, the microorganisms can be grown on a solid or semi-solid substrate, such as, for example, corn, wheat, soybean, chickpeas, beans, oatmeal, pasta, rice, and/or flours or meals of any of these or other similar substances.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum, and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium chloride, and/or sodium carbonate. These inorganic salts may be used independently or in a combination of two or more.

In some embodiments, additional acids and/or antimicrobials in the liquid medium before and/or during the cultivation process may be added. Antimicrobial agents or antibiotics are used for protecting the culture against contamination. Additionally, antifoaming agents may also be added to prevent the formation and/or accumulation of foam when gas is produced during cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the liquid medium may be necessary.

The method and equipment for cultivation of microorganisms and production of the microbial by-products can be performed in a batch, quasi-continuous, or continuous processes.

In one embodiment, the cultivation of microorganisms is carried out at about 5° C. to about 100° C., preferably, about 15° C. to about 60° C., more preferably, about 25° C. to about 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a microbe-free medium or contain cells, spores, mycelia, conidia, or other microbial propagules. In this manner, a quasi-continuous system is created.

In some embodiments, the glycolipids produced by microorganisms of interest may be retained in the microorganisms or secreted into their growth medium. The growth medium may contain compounds that stabilize the activity of the glycolipids. The glycolipids can be purified, or the glycolipids can be used in crude form, meaning they are not separated from the fermentation broth in which they were produced. In some embodiments, the crude form of glycolipids may contain microorganisms which produced the glycolipids. Such microorganism may be in an active or inactive form, or in the form of vegetative cells, reproductive spores, conidia, mycelia, hyphae, or any other form of microbial propagule. The crude form may also contain a combination of any of these forms of a microorganism.

In certain embodiments, the glycolipid is isolated and/or purified from the growth medium resulting from fermentation of a biosurfactant-producing microorganism. Isolation and purification can be easily achieved using standard methods or techniques described in the literature. The glycolipid can be further concentrated, if desired.

As used herein, the terms “isolated” or “purified,” when used in connection with biological or natural materials such as glycolipids means the material is substantially free of other compounds, such as cellular material, with which it is associated in nature. That is, the materials do not occur naturally without these other compounds and/or have different or distinctive characteristics compared with those found in the native material.

In certain embodiments, purified compounds are at least 60% by weight of the compound of interest. Preferably, the preparation is at least 75%, more preferably at least 90%, and most preferably at least 99% or 100% (w/w) of the desired compound by weight. Purity is measured by any appropriate standard method, for example, by column chromatography, thin layer chromatography, or high-performance liquid chromatography (HPLC) analysis.

The glycolipids according to the invention can be utilized in any of its structural forms, including derivatives, fractions, conformations, isoforms, subtypes, including forms that are naturally or artificially (or chemically) modified. The use of different isomers or forms of glycolipids is beneficial in that the skilled artisan can tailor the composition depending upon, for example, its effectiveness for reducing the occurrence of skin irritation or dryness. That is, certain isoforms of a glycolipid might be more effective to, for example, lessen the undesirable effects of certain removal solvents while retaining the removal efficiency of the removal solvents.

The degree of polarity of glycolipids depends on the hydrocarbons used as a substrate. Typical examples of glycolipids according to the present invention include rhamnolipids produced by, e.g., Pseudomonas spp.; sophorolipids produced by e.g., Candida spp., Starmerella bombicola, and/or Wickerhamomyces anomalus (e.g., NRRL Y-68030); trehalose lipids produced by Rhodococcus spp.; and mannosylerythritol lipids produced by Pseudozyma spp.

In certain embodiments, the glycolipid according to the present invention is a sophorolipid (SLP), such as, for example, a lactonic or acidic form sophorolipid, a non-acetylated sophorolipid, a mono-acetylated sophorolipid, a di-acetylated sophorolipid, or any other isoform thereof. SLPs are produced in large quantity by several nonpathogenic yeast species, the most studied of which is Starmerella bombicola. Some Pichia yeasts (e.g., P. anomala) are also capable of producing SLPs. SLPs have environmental compatibility, high biodegradability, low toxicity, high selectivity, and specific activity in a broad range of temperature, pH, and salinity conditions.

In certain embodiments, the glycolipid according to the present invention is a rhamnolipid (RLP), such as, for example, a mono-rhamnolipid, a di-rhamnolipid, or any other isoform thereof. RLPs are glycolipids produced mainly by Pseudomonas bacteria. RLPs can be used as antibacterial (Gram-positive) and antifungal agents.

In certain embodiments, the glycolipid according to the present invention is a mannosylerythritol lipid (MEL), such as, for example, MEL-A, MEL-B, MEL-C, or MEL-D, or any other isoforms with varying fatty acid lengths and/or hydrophobic portions. MELs are glycolipids produced mainly by the yeast genus Pseudozyma. MELs are non-toxic and are stable at wide temperatures and ph ranges. Furthermore, MELs can be used without any additional preservatives.

In certain embodiments, the glycolipid according to the present invention is a trehalose lipid (TL), including any form thereof. TLs are glycolipids produced by, for example, the bacteria Rhodococcus erythropolis. TLs possess emulsifying and dispersing characteristics. They exhibit increased levels of surface activity and have certain antiviral and antimicrobial properties.

The glycolipids formulated into a nail polish remover composition according to the present invention are capable of mitigating or lessening damages to the skin and/or nails caused by one or more ingredients in the nail polish remover composition, in particular, a removal solvent. As a result, the glycolipids present in the nail polish remover composition function to reduce the occurrence of dry nails as well as dry and/or irritated skin surrounding the nails compared to a nail polish remover without such glycolipids after the composition has contacted the nails and skin. Dry skin typically can be identified by its appearance and feel. Dry skin often has a parched or wrinkled look caused by the skin's inability to retain moisture. It often feels “tight” and uncomfortable, which may include pain or itchiness. It is prone to irritation and other skin conditions which may exhibit one or more of redness, roughness, blisters, bumps, chapping, flaking, cracking, or the like. Similarly, dry nails are typically characterized by its brittleness, which is prone to cracking, chipping, splitting, peeling, or the like.

Furthermore, the amphiphilic nature of glycolipids facilitates penetration or “wetting” of the nail care products to be removed by the nail polish remover composition, enabling a more efficient removal process. Thus, in addition to providing skin benefits, the glycolipids can enhance the overall effectiveness of the nail polish remover composition in its intended purpose to remove nail care products from bodily and non-bodily surfaces.

The glycolipid present in the nail polish remover composition according to the present invention can be one type of glycolipid or a combination of one or more types of glycolipids. For example, one embodiment of a nail polish remover composition according to the present invention comprises a sophorolipid and a mannosylerythritol lipid. Other embodiments may contain a sophorolipid only or a mannosylerythritol lipid only.

### Removal Solvent

The nail polish remover composition according to the present invention further comprises a removal solvent. In some embodiments, the removal solvent is present in the composition in about 50% by weight to about 99% by weight of the total composition, more preferably, 65% by weight to about 95% by weight of the total composition.

“Removal solvent” as used herein refers to an active ingredient of the nail polish remover composition of the present invention. It acts to solubilize, dissolve, weaken, loosen, or otherwise disrupt the substance that is part of a nail care product attached to or otherwise disposed on the user's nail. In some embodiments, the removal solvent is acetone, acetates, and/or methyl soyate. The acetate is preferably methyl acetate, ethyl acetate, and/or butyl acetate. In other embodiments, the removal solvent is an alcohol (such as isopropyl, methyl, or ethyl alcohol), propylene carbonate, a dibasic ester (such as dimethyl succinate, dimethyl glutarate, and/or dimethyl adipate), and/or a lactate such as ethyl lactate. Preferably, the removal solvent is acetone, methyl acetate, ethyl acetate, butyl acetate, and/or methyl soyate.

In other preferred embodiments, the removal solvent according to the present invention comprises a biologically produced removal solvent, which is a product of cultivation or fermentation of a microorganism. More preferably, the biologically produced removal solvent is an acetate such as methyl acetate, ethyl acetate, and/or butyl acetate. In some embodiments, a biologically produced acetate that is a fermentation product of microorganisms such as yeasts. In a specific embodiment, the yeasts are those characterized as non-pathogenic yeast strains such as Wickerhamomyces anomalus, or other yeasts related thereto, such as other members of the Wickerhamomyces and/or Pichia clades including Pichia guilliermondii (Meyerozyma guilliermondii), Pichia kudriavzevii, and/or Pichia occidentalis. Fermentation that results in the production of an acetate may be carried out according to a method described herein or any other method known in the art. A biologically produced acetate may be used in combination with, or in lieu of, any non-biologically produced removal solvents.

In one embodiment, the biologically produced acetate may be extracted from the fermentation medium and purified from other fermentation products containing the microorganisms and/or other microbial metabolites (e.g., enzymes such as phytase and exo beta-1,3-glucanase) produced by the microorganisms and/or any residual nutrients. If desired, extraction and purification can be easily achieved using standard extraction and/or purification methods or techniques described in the literature or known in the art. In other embodiments, the acetate may be used directly without extraction or purification.

The biologically produced acetate according to the present invention can be a mixture of two or more acetates (e.g., ethyl acetate and methyl acetate) or a single type of acetate (e.g., ethyl acetate). The microorganism as a source of the acetate may be one species (e.g., Wickerhamomyces anomalus), or two or more species that are cultivated either together or separately. In some embodiments, the biologically produced acetate of the present invention may be produced by the same species or strain that produced the glycolipid of the present invention. In other embodiments, the biologically produced acetate of the present invention is produced by a different species or strain than that produced the glycolipids of the present invention.

In some embodiments, the biologically produced acetate comprises ethyl acetate produced by fermentation of Wickerhamomyces anomalus. Other closely-related species are also envisioned, including other members of the Wickerhamomyces and/or Pichia clades, e.g., Pichia guillicrmondii (Meyerozyma guilliermondii), Pichia kudriavzevii, and/or Pichia occidentalis. In specific embodiments, the strain of Wickerhamomyces anomalus that is used to produce ethyl acetate is NRRL Y-68030. Ethyl acetate produced by Wickerhamomyces anomalus (“Wicker's ethyl acetate”) may be used in combination with, or in lieu of, any biologically or non-biologically produced removal solvents. For example, Wicker's ethyl acetate may be used as the sole removal solvent in a nail polish remover composition according to the present invention. In other examples, Wicker's ethyl acetate may be used in combination with a non-biologically produced removal solvent such as acetone.

Glycol Co-Solvent In some embodiments, the nail polish remover composition further comprises one or more glycol co-solvent. In certain embodiments, the glycol co-solvent is present in the nail polish remover composition of the present invention in up to about 20% by weight of the total composition.

The glycol co-solvent is an alcohol that is liquid at room temperature and possesses two or more hydroxyl functional groups attached to carbon atoms of an alkyl chain. The glycol co-solvent enhances the effects of glycolipids in the nail polish remover composition by protecting the skin and/or nails from irritants and dryness. The glycol helps to attract moisture and mitigate the harsh effects of removal solvents.

Preferably, the alkyl chain of the glycol co-solvent has between C3 and C5 in length. More preferably, the glycol co-solvent comprises a C3 or C4 alkyl chain. In certain embodiments, the glycol co-solvent is 1,3-butylene glycol, 1,3-propanediol, and/or glycerol.

### Water and Other Components

In some embodiments, the nail polish remover composition comprises water. In some embodiments, water is present in up to about 10% by weight of the total nail polish remover composition.

In further embodiments, the nail polish remover composition according to the present invention optionally comprises additional components known in the art. In some embodiments, the nail polish remover composition does not include such optional components. Preferably, such components are non-toxic for topical application and do not adversely interfere with the functions of the removal solvent or glycolipids. The additional components may include, but are not limited to, oils, fragrance, colorants, and thickeners. In certain embodiments, the additional components are selected from commercially available ingredients approved for food, drug, and/or cosmetic applications.

In some embodiments, the nail polish remover composition comprises no biosurfactant other than glycolipids. In other embodiments, the nail polish remover composition comprises a biosurfactant other than the glycolipids as described herein. Such other biosurfactants include, but are not limited to, lipopeptides (e.g., surfactin, iturin, fengycin, arthrofactin and lichenysin), and phospholipids (e.g., cardiolipin).

In certain embodiments, the additional biosurfactant is a lipopeptide, including linear or cyclic form lipopeptides, or any other isoforms thereof. As an example, surfactin is a lipopeptide that can have a structure comprising a peptide loop of seven amino acids and a hydrophobic fatty acid chain thirteen to fifteen carbons long. In an exemplary embodiment, the amino acids comprise L-aspartic acid, L-leucine, glutamic acid, L-leucine, L-valine and two D-leucines.

As another example, iturin is a lipopeptide with a structure comprising a peptide loop of seven amino acids and a β-amino fatty acid chain that can vary from 14 to 17 carbons long. In one embodiment, iturin A is utilized according to the subject invention.

One of skill in the art can select additional components and determine suitable amounts and formulations such that the final composition functions with the desired degree of effectiveness to remove a nail care product as provided herein.

In accordance with certain embodiments of the present invention, the nail polish remover composition is topically applied to a bodily surface, preferably fingernails, toenails, and/or the surrounding skin. In some embodiments, the nail polish remover composition may be packaged in a container from which the user dispenses a desired amount of the composition. An absorbent material such as a cotton ball or pad, felt pad, cloth, sponge, or the like that can absorb the composition may be used as a vehicle to transfer the composition from the container to the intended surface. Other non-limiting, exemplary packages for the nail polish remover composition include: a set of a container filled with the composition provided with a separate container of dry cotton balls or pads; individual felt or cotton pads already soaked in the composition; and a container filled with foam or sponge soaked with the nail polish remover composition into which the user inserts a finger, and the container is subsequently twisted until the nail care product comes off of the finger. Variations in the packaging of the nail polish remover composition are within the purview of a skilled artisan.

### Treatment Composition

In another aspect, the present invention provides treatment compositions and methods of their use. In particular, the subject invention provides topical compositions that can treat dry nails and surrounding skin and/or a fungal infection of hands and feet. In some embodiments, the treatment composition is used alone, concomitantly with, or otherwise close in time to, the application of the nail polish remover composition.

In certain embodiments, the treatment composition comprises biochemical-producing microorganisms, with or without by-products of their growth, such as biosurfactants, solvents and/or enzymes. In one embodiment, the composition comprises biochemical-producing microorganisms as well as the fermentation broth resulting from the cultivation thereof. The treatment composition may be, for example, by weight, at least 0.01%, at least 0.05%, at least 0.1%, at least 0.5%, at least 1%, at least 2%, at least 5%, at least 10%, at least 25%, at least 50%, at least 75%, or 100% (w/w) of the broth including the biochemical-producing microorganisms.

In certain embodiments, the compositions of the subject invention have advantages over, for example, biosurfactants alone, including one or more of the following: high concentrations of mannoprotein as a part of yeast cell wall's outer surface; the presence of biopolymer beta-glucan in yeast cell walls; the presence of biosurfactants in the culture, which are capable of reducing both surface and interfacial tension; and the presence of metabolites (e.g., lactic acid, ethanol, etc.).

In other embodiments, purified biochemical-producing microorganisms are provided in the composition with minimal to no by-products of their growth. The treatment composition may be, for example, by weight, at least 0.01%, at least 0.05%, at least 0.1%, at least 0.5%, at least 1%, at least 2%, at least 5%, at least 10%, at least 25%, at least 50%, at least 75%, or 100% (w/w) of the purified biochemical-producing microorganisms.

In preferred embodiments, the biochemical-producing microorganism of the subject compositions is a cultivated biosurfactant-, solvent-and/or enzyme-producing yeast. In one embodiment, the microbe is, for example, cultivated Starmerella bombicola, Candida apicola, Wickerhamomyces anomalus (e.g., NRRL Y-68030), Pichia guilliermondii, Pichia occidentalis, and/or Pseudozyma aphidis. In one embodiment the microbe is a cultivated mutant of any of these species of yeast.

If desired, the treatment compositions can have a high density of the biochemical-producing microorganism in the form of vegetative cells, inactivated cells, or a mixture of vegetative cells, inactivated cells, reproductive spores, mycelia and/or other microbial propagules. The treatment composition according to some embodiments comprises a biochemical-producing microorganism predominantly (more than about 50%) in an inactivated form. In other embodiments, the treatment composition comprises a biochemical-producing microorganism predominantly in an active form.

In certain embodiments, the treatment composition comprises microbial growth by-products with or without biochemical-producing microorganisms. In certain embodiments, the microbial growth by-product is provided in the composition with the biochemical-producing microorganism that produced it and/or a different biochemical-producing microorganism. In some embodiments, a purified microbial growth by-product can be included in the composition.

In some embodiments, microbial growth by-products include enzymes such as exo-beta-1,3-glucanase, esterases, lipases, glycosidases, amylases, proteases, and chitinases. In preferred embodiments, the microbial growth by-product of the subject compositions is a biosurfactant, solvent, enzyme or other metabolite. The microbial growth by-product of the present invention can be derived from the same biochemical-producing microorganism(s) or a mixture of microbial growth by-products from two or more different biochemical-producing microorganisms. In one embodiment, growth by-products produced by the microorganism work synergistically with one another to produce a desired effect.

Particularly, the growth by-products according to the invention comprise biosurfactants. In some embodiments, the biosurfactants are utilized in a crude form, wherein the molecule is present in the broth in which the microorganism is cultivated and is collected therefrom without purification. The crude form can comprise, for example, at least 20%, 30%, 40%, 50%, 60%, 70% or 80% biosurfactant molecule in broth. In some embodiments, the biosurfactants have been purified from the products of cultivation.

The biosurfactants according to the present invention include glycolipids as described herein, such as mannosylerythritol lipids (MELs), sophorolipids (SLPs), trehalose lipids (TLs), and rhamnolipids (RLP), including any subtypes and isoforms thereof. Other biosurfactants that can be included in the composition include lipopeptides, such as surfactin, iturin such as iturin A, fengycin, arthrofactin, and lichenysin; and/or phospholipids such as cardiolipin. In one embodiment, the composition can comprise a combination of any of these biosurfactants.

In certain embodiments, the treatment composition comprises a therapeutically effective amount of biosurfactants. In preferred embodiments, biosurfactant concentration in the treatment composition ranges from 0.001% to 90% of the total composition by weight, from 0.01% to 50%, from 0.05% to 10%, from 0.1% to 5.0%, and from 0.01% to 2.0%. In certain embodiments, the biosurfactant is present at more than 0.01, 0.02, 0.03, 0.05, 0.08, 0.1, 0.2, or 0.5%.

In some embodiments, the treatment composition of the present invention does not utilize a non-biologically produced removal solvent as an active component of the composition. In other embodiments, the treatment composition of the present invention does not utilize a biologically produced removal solvent as an active component of the composition. In yet other embodiments, the treatment composition of the present invention does not utilize any removal solvent as an active component.

In other embodiments, the treatment composition according to the present invention includes a biochemical-producing microorganism and/or a microbial by-product, and nothing else that materially affects the hydrating and/or anti-fungal effects of the treatment composition. In some embodiments, the treatment composition includes a biochemical-producing microorganism and/or a microbial by-product as well as a glycol co-solvent, and nothing else that materially affects the hydrating and/or anti-fungal effects of the treatment composition. In certain embodiments, a biochemical-producing microorganism and/or a microbial by-product work synergistically with a glycol co-solvent to produce a desired effect.

In yet other embodiments, the treatment composition according to the present invention comprises a glycol co-solvent as described herein in addition to a biochemical-producing microorganism and/or a microbial by-product, and any other components that are suitable for use on the skin and nails, provided that they do not adversely interfere with the hydrating and/or anti-fungal effects of the treatment composition. In certain embodiments, such additional components are selected from commercially available and/or otherwise known ingredients approved for food, drug, and/or cosmetic applications.

In some embodiments, the treatment composition further comprises a topical antifungal medication such as Ageratina pichinchensis (snakeroot) extract, Melaleuca alternifolia (tea tree) oil, cyanoacrylate, undecylenic acid, and hydroquinone, menthol, ciclopirox, miconazole, and clotrimazole. In one embodiment, biochemical-producing microorganisms and/or microbial by-products (e.g., biosurfactant) works synergistically with an antifungal medication in treating a fungal infection of the area to the which the treatment composition is applied.

In other embodiments, the treatment composition comprises biochemical-producing microorganisms and/or microbial by-products (e.g., biosurfactant) as the only active ingredient(s) in the composition for treating a fungal infection.

### Methods of Use and Skin Protection

In another aspect, the present invention relates to a method of removing a nail care product from a bodily or non-bodily surface with a nail polish remover composition comprising a glycolipid and a removal solvent. In some embodiments, the nail care product can be nail polish, artificial nails, or both.

In some embodiments, the nail polish remover composition comprising a glycolipid and a removal solvent can be applied to a desired surface by absorbing an appropriate amount of the composition onto an absorbent material and rubbing the surface with it until the nail care product is removed. The absorbent material is then disposed of or washed for later use.

In alternative embodiments, an absorbent material soaked with the nail polish remover composition is placed on a desired surface and optionally covered with a non-absorbent material such as tin or aluminum foil to keep the absorbent material in place. In other embodiments, a nail care product attached to a surface (e.g., a fingernail painted with nail polish) is submerged directly into the nail polish remover composition or a foam/sponge soaked with such composition. In any of these methods described herein, the purpose is to allow an effective amount of the nail polish remover composition comprising a glycolipid and a removal solvent to come in direct contact with the nail care product to be removed, such that the composition sufficiently penetrates the nail care product. An “effective amount” as used herein refers to the amount of the composition sufficient to produce the primary intended result, which is removal of a nail care product from a surface.

In other embodiments according to the present invention, the presence of glycolipids in the nail polish remover composition facilitates efficient “wetting” of the nail care products to be removed by the nail polish remover composition, enabling a more efficient removal process compared to conventional nail polish removers.

Some embodiments of the present invention are directed to methods of protecting skin and/or nails of a subject from damaging effects of a removal solvent. The methods involve contacting the skin and nails with a composition comprising the removal solvent and a glycolipid according to the invention. The methods reduce the drying and/or irritating effects of removal solvents on the skin and/or nails, thereby reducing the occurrence of dry, irritated skin and/or dry nails after use of the nail polish remover composition.

In some embodiments, the present invention is used to hydrate nails and surrounding skin to which the nail polish remover composition is applied. In further embodiments, application of the nail polish remover composition according to the invention increases the moisture content of the area to which the nail polish remover composition was applied, compared to the moisture content of the same area immediately prior to the application.

Similarly, the present invention provides a method of hydrating nails and surrounding skin comprising applying a treatment composition according to the present invention. In further embodiments, application of the treatment composition according to the invention increases the moisture content of the area to which the treatment composition was applied, compared to the moisture content of the same area immediately prior to the application.

Moisture content of a skin or a nail before and after the usage of a nail polish remover composition or a treatment composition according to the present invention may be measured and/or determined by methods known in the art, including resistance and capacitance measurements with corneometer or Near infrared spectroscopy (NIR)-based prediction of water content.

In the method of hydrating nails and surrounding skin according to the present invention, the nail polish remover composition and the treatment composition may be applied alone (meaning only one of the compositions is used), separately, concomitantly, or otherwise close in time to each other. If both compositions are used on the same area of nail and/or skin, the treatment composition is preferably applied after and close in time to the application of the nail polish remover composition.

In other embodiments, the present invention provides a method of treating a fungal infection in feet and/or hands of a subject. In some embodiments, the fungal infection is onychomycosis, or nail fungus. When a dermatophyte causes toenail fungus, the condition is called tinea unguium. The method of treating onychomycosis, including tinea unguium, comprises topically applying an effective amount of the treatment composition to the affected area. In some embodiments, the method according to the invention treats onychomycosis caused by Trichophyton rubrum which is believed to be by far the most frequently isolated dermatophyte, or T. mentagrophytes, or both. In some embodiments, the treatment method can be combined with other onychomycosis treatments known in the art, including separate topical and oral treatments. In certain embodiments the method according to the present invention works synergistically with a known treatment to treat onychomycosis in a subject.

In yet other embodiments, the fungal infection that is treated according to the present invention is tinea manuum and tinea pedis (athlete's foot). The method of treating tinea manuum and tinea pedis comprises topically applying an effective amount of the treatment composition to the affected area in a subject. In some embodiments, the treatment method can be combined with other treatments of tinea manuum and/or tinea pedis known in the art, including separate topical and oral treatments. In certain embodiments the method according to the present invention works synergistically with a known treatment to treat tinea manuum and/or tinea pedis in a subject.

Other features and advantages of the invention will be apparent from the following description of the embodiments thereof, and from the claims. All references cited herein are hereby incorporated by reference.

### EXAMPLES

Following are Examples which are offered by way of illustration and are not intended to limit the invention. Unless otherwise stated, these Examples utilized the methods, techniques, and materials known in the art. Various modifications or changes in light thereof will be suggested to persons skilled in the art and are to be included within the spirit and purview of this application.

### Example 1: Compositions of the Nail Polish Remover According to the Present Invention

The exemplary formulations of the nail polish remover composition according to the present invention are presented herein as illustrations of various components that can be combined. The percentage values in the table below are weight percentages, based on the total weight of the composition.

TABLE 1

| Component | | Percentage |
|---|---|---|
| Functions | Component Options | (% w/w) |
| Removal | acetone, methyl acetate, ethyl acetate, | 65%-95% |
| solvent | butyl acetate, and/or methyl soyate | |
| Glycolipids | Sophorolipids and/or | 1%-5% |
| | mannosylerythritol lipids | |
| Glycol | 1,3-butylene glycol, 1,3-propanediol, | 0%-20% |
| co-solvent | and/or glycerol | |
| Water | Water | 0%-10% |
| solvent | | |

### Example 2: Fermentation of Starmerella Bombicola for SLP Production and Purification

Fermentation of Starmerella bombicola for SLP production was performed in a 14-liter reactor as a fed-batch fermentation in a nutrient medium containing 0.5% yeast extract, 10% glucose, 10% canola oil, 1% urea. The initial pH was 5.5. Temperature of cultivation was 25° C.

Initially, fermentation continued for 5 days and then resulting SLP was harvested. After adding additional amounts of the nutritional components into the reactor, keeping the same proportions, the process continued. Then, in 2-3 days, new portions of SLP were harvested.

The amount of SLP harvested from each cycle ranged from 0.5-1.0 liters of the final product. Concentration of SLP in the product typically reached 50%. This quasi-continuous technological process typically continued for 2 weeks, after which a new cycle would begin.

After SLP was harvested from the fermentation products, the SLP was purified by conducting filtration of 10% water solution through the filters with 2-micron pore size. The product could then be stored in a refrigerator for making the nail polish remover composition.

### Example 3: Fermentation of Pseudozyma Aphidis for Mannosylerythritol Lipid (MEL) Production

Fermentation of Pseudozyma aphidis for MEL production was performed in Portable 14 L Distributable Reactor, which is a steam autoclavable jacketed glass vessel with air spurge and Rushton impeller. It was equipped with dissolved oxygen (DO), pH, temperature, and foam probe. It had an integrated control station with a color touchscreen interface, built-in pumps, gas flow controllers, and pH/DO foam/level controllers. The working volume of the reactor was 10 liters.

The nutrient medium composition comprised sodium nitrate, potassium phosphate, magnesium sulfate, yeast extract, and vegetable oil. Inoculum was a 1- to 2-day old culture of Pseudozyma aphidis, at about 5-10% of the total culture volume. The duration of cultivation duration and sample collection for each run was 9-15 days. Final MEL production varied between 800-1,000 grams. The product could then be stored in a refrigerator for making the nail polish remover composition.

### Example 4: Production of a Treatment Composition

An embodiment of a treatment composition according to the invention is obtained using a medium containing glucose, canola oil, yeast extract, NH₄Cl, KH₂PO₄·H₂O and MgSO₄·7H₂O. The initial pH is adjusted to about 5.5 with KOH. The cultures are grown at about 25° C. with no stabilization.

Wickerhamomyces anomalus and Pseudozyma aphidis are cultured in different tanks. After completion of the fermentation process, the composition is prepared by inactivation of the culture at pasteurization temperature (up to 65° to 70° C. for a time period sufficient to inactivate 100% of the yeast cells) and increasing pH value up to about 10.0-12.0. This induces partial hydrolysis of cells and allows for freeing of some nutritional components therein. Then, the composition is neutralized to a pH of about 6.5-7.5 and the various components of hydrolysis are mixed.

1. A nail polish remover composition comprising a removal solvent and a glycolipid.

2. The nail polish remover composition of claim 1, wherein the removal solvent is acetone, acetate, and/or methyl soyate.

3. The nail polish remover composition of claim 1, comprising a glycolipid selected from rhamnolipids, a sophorolipids, mannosylerythritol lipids, and trehalose lipids.

4. The nail polish remover composition of claim 3, comprising a sophorolipid and/or a mannosylerythritol lipid.

5. The nail polish remover composition of claim 1, further comprising a glycol co-solvent.

6. The nail polish remover composition of claim 5, comprising a glycol co-solvent selected from 1,3-butylene glycol; 1-3-propanediol; and glycerol.

7-8. (canceled)

9. The nail polish remover composition of claim 1, wherein the composition comprises between about 1% by weight and about 5% by weight of said glycolipid.

10-11. (canceled)

12. A nail polish remover composition comprising:
about 1% by weight to about 5% by weight of a glycolipid;
about 65% by weight to about 95% by weight of a removal solvent;
about 0% by weight to about 20% by weight of a glycol co-solvent; and
about 0% by weight to about 10% by weight of water.

13. The nail polish remover composition of claim 12, comprising a glycolipid selected from sophorolipids and mannosylerythritol lipids.

14. The nail polish remover composition of claim 12, comprising a glycol co-solvent selected from 1,3-butylene glycol; 1-3-propanediol; and glycerol.

15. The nail polish remover composition of claim 12, wherein the removal solvent is acetone, acetate, and/or methyl soyate.

16. A method of removing a nail care product from a surface comprising: applying an effective amount of a composition according to claim 1 to the surface; and removing the nail care product from the surface.

17. The method of claim 16, wherein the composition further comprises a glycol co-solvent.

18. The method of claim 16, wherein the composition comprises a glycolipid selected from sophorolipids and mannosylerythritol lipids.

19. The method of claim 16, wherein the composition comprises:
about 1% by weight to about 5% by weight of the glycolipid;
about 65% by weight to about 95% by weight of the removal solvent;
about 0% by weight to about 20% by weight of a glycol co-solvent; and
about 0% by weight to about 10% by weight of water.

20. A method of protecting skin surrounding nails of a subject from damaging effects of a removal solvent comprising:
contacting said skin of the subject with the nail polish remover composition of claim 1.

21. A method of hydrating a nail and skin surrounding the nail of a subject comprising:
Providing at least one of a nail polish remover composition of claim 1 and a treatment composition,
applying to said nail and said skin surrounding the nail said at least one of the nail polish remover composition or the treatment composition,
wherein said treatment composition comprises a biochemical-producing microorganism and/or a microbial by-product, and
wherein the moisture content of said nail and said skin surrounding the nail is higher after said application compared to immediately before said application.

22. The method of claim 21, wherein said treatment composition is applied to said nail and said skin surrounding the nail immediately after said nail polish remover composition is applied.

23. A method of treating a fungal infection of feet or hands of a subject, comprising:
applying an effective amount of a treatment composition to the subject in need of such treatment;
wherein said treatment composition comprises a biosurfactant.

24. The method according to claim 21, wherein said fungal infection of feet or hands is onychomycosis, tinea pedis, or tinea manuum.
